(19) United States
(12) Patent Application Publication
(10) Pub. No.: US 2008/0134361 A1
(43) Pub. Date: Jun. 5, 2008

(54) Grain Quality Through Altered Expression of Seed Proteins

(75) Inventor: Rudolf Jung, Des Moines, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/782,965

(22) Filed: Jul. 25, 2007

Related U.S. Application Data
(63) Continuation-in-part of application No. 11/546,627, filed on Oct. 12, 2006.
(60) Provisional application No. 60/728,784, filed on Oct. 20, 2005.

(51) Int. Cl. A01H 1/02; C12N 15/11; A01H 5/00

(52) U.S. Cl. 800/286; 536/24.5; 800/298; 800/320; 800/312; 800/278; 536/23.5

(57) ABSTRACT

The present invention is directed to compositions and methods for altering the levels of seed proteins in seed or grain. The invention is directed to the alteration of seed protein levels in plants, resulting in grain and seed with increased digestibility/nutrient availability, improved amino acid composition/nutritional quality, increased response to feed processing, improved silage quality, and increased efficiency of wet milling. The invention is further directed to nucleotide sequences encoding a sorghum delta-kafirin2 protein, sequences encoding a sugar cane delta prolamin2 protein, sequences encoding a sorghum LKR protein, and the amino acid sequences so encoded. Methods of using such sequences are also provided.

### CROSS-REFERENCE TO RELATED APPLICATIONS

This application is a continuation-in-part of co-pending application U.S. application Ser. No. 11/546,627 filed Oct. 12, 2006 which claims priority to, and the benefit of, U.S. Provisional Application Ser. No. 60/728,784 filed Oct. 20, 2005, the contents of which are incorporated herein by reference.

### FIELD OF THE INVENTION

The invention relates to the field of plant molecular biology and the use of genetic modification to improve the quality of crop plants, more particularly to methods for improving the nutritional value of seed and grain and the efficiency of grain processing.

### DETAILED DESCRIPTION OF THE INVENTION

The present invention is directed to compositions and methods for altering the levels of seed proteins in plant seed, particularly reducing the levels of kafirin in sorghum. Modification of seed protein composition causes changes in the physical and/or chemical properties of the seed.

Sorghum (Sorghum bicolor), one of the most important staple crops in Africa, represents the fifth most important cereal crop in the world. It is the only viable food grain for many of the world's most food insecure people, and can make critically important contributions to nutrition of families and children affected by AIDS and other pandemics.

Sorghum grain has a nutritional profile similar to corn and other cereals (Shewry and Halford, Journal of Experimental Botany 53 (570):947-958 (2002), i.e. it shares the typical nutritional deficiencies of cereal grains, a low content of the essential amino acids lysine, threonine, tryptophan and sulphur amino acids; and a low bio-availability of iron and zinc. Therefore, a diet, based mostly on sorghum, is not adequate to meet the nutritional growth or maintenance requirements for children and adults and needs to be supplemented with essential amino acids and micronutrients. Further, most sorghum food is cooked or heated during preparation. In contrast to other cereal grains, heat treatment results in a severely reduced digestibility of sorghum grain (up to 50%). The high cysteine content of kafirins is considered to contribute to disulfide-bridge related cross-linking of seed proteins during seed development. By repressing delta-kafirin2 expression (SEQ ID NO: 1), the potential for protein cross-linking is reduced and digestibility of sorghum subjected to heat is improved.

The invention is directed to the alteration of protein composition and levels in plant seed, resulting in grain or seed with increased digestibility, increased energy availability, improved amino acid composition, improved nutritional value, increased response to feed processing, improved silage quality, increased efficiency of wet or dry milling, and decreased anti-nutritional properties. The claimed sequences encode proteins preferentially expressed during seed development.

Typically, “grain” means the mature kernel produced by commercial growers for purposes other than growing or reproducing the species, and “seed” means the mature kernel used for growing or reproducing the species. For the purposes of the present invention, “grain”, “seed”, and “kernel”, will be used interchangeably.

As used herein, “genetically modified” or “genetically altered” means the modified expression of a seed protein resulting from one or more genetic modifications; the modifications including but not limited to: recombinant gene technologies, induced mutations, and breeding stably genetically modified plants to produce progeny comprising the altered gene product.

Compositions of the invention comprise sequences encoding plant seed proteins and variants and fragments thereof. Methods of the invention involve increasing or inhibiting a seed protein by such means as, but are not limited to, transgenic expression, antisense suppression, co-suppression methods including but not limited to: RNA interference, gene activation or suppression using transcription factors and/or repressors, mutagenesis including transposon tagging, directed and site-specific mutagenesis, chromosome engineering (see Nobrega et. al., Nature 431:988-993(04)), homologous recombination, TILLING (Targeting Induced Local Lesions In Genomes), and biosynthetic competition to manipulate, in plants and plant seeds and grains, the expression of seed proteins, including, but not limited to, those encoded by the sequences disclosed herein.

Transgenic plants producing seeds and grain with altered seed protein content are also provided.

The genetically modified seed and grain of the invention can also be obtained by breeding with transgenic plants, by breeding between independent transgenic events, by breeding of plants with one or more alleles (including mutant alleles) of genes encoding abundant seed proteins and by breeding of transgenic plants with plants with one or more alleles (including mutant alleles) of genes encoding abundant seed proteins. Breeding, including introgression of transgenic and mutant loci into elite breeding germplasm and adaptation (improvement) of breeding germplasm to the expression of transgenes and mutant alleles, can be facilitated by methods such as marker assisted selected breeding.

It is recognized that while the invention is exemplified by the modulation of expression of selective sequences in sorghum and sugarcane, similar methods can be used to modulate the levels of seed proteins in other plants: in particular cereal plants such as millets, rice and maize. In other embodiments, the methods can be used to express and accumulate the seed proteins of the invention in other plants such as alfalfa, soybean and cassaya.

Soybean, like other legumes, when used as feed is deficient in the sulfur amino acids (methionine, cysteine). Animal diets based on soybean and soy products, such as meal, are typically fortified with methionine to achieve optimal nutritional balance.

Attempts to genetically modify soybeans to enrich for sulfur rich proteins have been problematic. Ectopically expressed proteins with high contents of sulfur amino acids did not accumulate to high levels because of instability or in other cases, led to allergenicity. Therefore, there is a need for methods for increasing the sulfur amino acids in soybeans. Both the sorghum delta-kafirin2 (SEQ ID NO: 1), and sugarcane delta prolamine 2 (SEQ ID NO: 3) nucleic acid sequences can be provided in expression cassettes with suitable promoters for transformation into soybeans and can be expected to provide improved nutritional quality (i.e., improved amino acid composition) over wild-type soybean.

Here a novel sorghum kafirin has been isolated and identified as sorghum delta-kafirin2, the nucleotide sequence shown at SEQ ID NO: 1, and the amino acid sequence at SEQ ID NO: 2. A highly homologous protein and cDNA to this sequence was also isolated from sugar cane, herein called delta-prolamin2, the nucleotide sequence set forth at SEQ ID NO: 3, and the amino acid sequence at SEQ ID NO: 4. As with zeins, these amino acids are very rich in cysteine residues, and suppression in sorghum endosperm is expected to result in sorghum grain with improved digestibility.

The sequences of the invention can be used to identify and isolate similar sequences in other plants based on sequence homology or sequence identity. Alternatively, where the sequences of the invention share sufficient homology to modulate expression of the native genes, the sequences can be used to modulate expression in other plants.

In sorghum, the prolamins are referred to as kafirins, which commonly, but not necessarily, share a high homology to the zeins of maize. See, for example DeRose R T, et al, “Characterization of the kafirin gene family from sorghum reveals extensive homology with zein from maize” Plant Mol. Biol. 12(3): 245-256 (1989). Expression of kafirins in maize has been demonstrated. Song, R. et al. “Expression of the sorghum 10-member kafirin gene cluster in maize endosperm” Nuc. Acids Res. Vol. 32, No. 22: e189, 1-8 (2004). For a review of sorghum seed proteins including kafirin see Leite et al., The Prolamins of Sorghum, Coix and Millets., In: Shewry and Casey (eds.) (1999) Seed Proteins, 141-157, Academic Publishers, Dordrecht. For a review of sorghum seed protein structure and functionality see Belton et al., Kafirin structure and functionality, Journal of Cereal Science 44 (2006) 272-286.

The invention provides methods for increasing the lysine content of sorghum grain over wild-type by down regulation of a novel sorghum LKR nucleic acid (SEQ ID NO: 6) encoding a 1,060 amino acid protein (SEQ ID NO: 7). Expression cassettes, transgenic plants, seeds and method of using the sorghum LKR is herein disclosed.

In cereals, a major group of seed proteins is prolamins. Prolamins are typically characterized by being extractable in 70% ETOH and a reducing agent (see Woo et al., 2001, Plant Cell 13:2297-2317, and Shewry and Casey (eds.) (1999) Seed Proteins 141-157, Academic Publishers, Dordrecht.). However, prolamins can also be identified phylogenetically through the use of sequence analysis. Zeins are a type of prolamin seed protein found in maize. Kafirins are a type of prolamin seed protein found in sorghum. For the different classes of prolamin proteins in sugar cane, no special names have been in use commonly in the literature. Here they are referred to as sugar cane alpha-prolamin, sugar cane beta-prolamin, sugar cane gamma prolamin, sugar cane delta-prolamin, and so on, to indicate their relatedness to corresponding prolamin classes in maize in sorghum.

Other abundant seed proteins in cereal crops include, but are not limited to, the globulin proteins. The globulin proteins include, but are not limited to legumin and alpha-globulins. The corn and sorghum legumins and the corn and sorghum alpha-globulins are examples of globulins that are minor seed proteins in maize and sorghum; the name designation of both proteins are based on their phylogentic relationship to seed proteins from other species (Woo et al.).

Seed proteins have been traditionally characterized based on solubility characteristics (Shewry and Casey (eds.) (1999) Seed Proteins, 141-157, Academic Publishers, Dordrecht). Thus, most seed proteins are either extractable in aqueous alcoholic solutions (prolamins), extractible in aqueous solutions of low ionic strength (albumins), or extractable in aqueous solutions of high ionic strength (globulins).

The classification of seed proteins by extraction methods is well known in the art (Shewry and Casey (1999)). However, it is also common to designate seed proteins with unknown extraction characteristics as globulins, albumins, or prolamins if they are phylogenetically or sequence-related to proteins that have originally been classified based on extraction experiments. Therefore, it is a common practice to name seed proteins based on their phylogenetic association rather then their extraction properties. The name of a seed protein gene may therefore not reflect the properties of the encoded protein in a strict sense.

It has been recently discovered that down-regulation or inhibition of the prolamin proteins (including kafirin proteins) alone, or in combination, increases digestibility and the energy availability of cereal grain such as corn and sorghum.

Additionally, the novel discovery has been made that the up-regulation (or overexpression) of the non-zein proteins increases digestibility of cereal grain.

In one embodiment of the invention, kafirin proteins are down-regulated in combination with down-regulation of sorghum lysine ketoglutarate reductase (LKR) to produce grain with an elevated level of free lysine as well as improved digestibility.

The present invention also provides isolated nucleic acid molecules comprising a nucleotide sequence encoding a Sorghum bicolor protein, herein designated as delta-kafirin2, having the nucleotide sequence of SEQ ID NO: 1, and the amino acid sequence of SEQ ID NO:2, as well as the highly homologous nucleotide sequence from sugar cane, Saccharum officinale, herein referred to as delta-prolamin2, SEQ ID NO: 3, the corresponding amino acid sequence of SEQ ID NO: 4.

By “decreased” and “increased” is intended that the measurement of a parameter is changed by at least 10%, 20%, 30%, 40%, 50%, 60%, 70%, 80%, 90%, 100%, 200% or more when compared to the measurement of that parameter in a suitable control.

The present invention also provides isolated nucleotide sequences comprising transcriptional units for gene over-expression and gene-suppression that have been used either as single units or in combination as multiple units to transform plant cells.

As used herein in connection with abundant seed proteins, “biologically active” means a protein that folds, assemble and interacts with other proteins, is available as a nitrogen source for seed germination and accumulates (ie: synthesis exceeds deposition) during seed development.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. “Functional fragments” of a nucleotide sequence may encode protein fragments that retain the biological activity of the targeted gene. Alternatively, fragments of a nucleotide sequence that are useful as hybridization probes generally do not encode fragment proteins retaining biological activity. Fragments of a nucleotide sequence that are useful for generating cells, tissues or plants transiently or permanently suppressing a gene or genes may not encode fragment proteins retaining biological activity. Fragments may be in sense or antisense or reverse orientation or a combination thereof. Thus, for example, fragments of such nucleotide sequence may range from at least about 10 nucleotides, at least about 20 nucleotides, about 50 nucleotides, about 100 nucleotides, and up to the full-length nucleotide sequence-encoding native sorghum kafirin and sugarcane prolamin proteins of the invention.

Fragments of the nucleotide sequences of the invention (SEQ ID NO: 1, or SEQ ID NO: 3) that encode a biologically active portion of the sorghum delta-kafirin2 (SEQ ID NO: 2) or sugar cane delta prolamin2 (SEQ ID NO: 4) respectively, will encode at least 15, 25, 30, 50, 100, 150, or 200 contiguous amino acids, or up to the total number of amino acids present in the full-length sorghum delta-kafirin2 or sugar cane prolamin2 of the invention (191 amino acids for SEQ ID NO: 2; and 178 amino acids for SEQ ID NO: 4). Fragments of SEQ ID NO: 1, or SEQ ID NO: 3 that are useful as hybridization probes or PCR primers need not encode a biologically active portion of a kafirin or prolamin protein.

Thus, a fragment of SEQ ID NO: 1 or SEQ ID NO: 3 may encode a biologically active portion of a prolamin or kafirin protein, or it may be a fragment that can be used as a hybridization probe or PCR primer using methods disclosed below or it may be used to inhibit the expression of the protein. A biologically active portion of the sorghum delta-kafirin2 or sugarcane delta-prolamin2 proteins of the invention can be prepared by isolating a portion of the disclosed nucleotide sequence that codes for a portion of the protein (e.g., by recombinant expression in vitro), and assessing the activity of the encoded portion of the prolamin protein. Nucleic acid molecules that are fragments of SEQ ID NO: 1 comprise at least 40, 50, 75, 100, 150, 200, 250, 300, 350, 400, 450, 500, 550, or 600 nucleotides, or up to the number of nucleotides present in the full-length sorghum delta-kafirin2 cDNA (for example, 649 nucleotides for SEQ ID NO: 1). Nucleic acid molecules that are fragments of SEQ ID NO: 3 comprise at least 40, 50, 75, 100, 150, 200, 250, 300, 350, 400, 450, 500, 550, 600, 650, 700, 800, nucleotides, or up to the number of nucleotides present in the full-length sugarcane delta prolamin2 cDNA (for example, 903 nucleotides for SEQ ID NO: 4).

By “variants” is intended substantially similar sequences. For nucleotide sequences, conservative variants include those sequences that, because of the degeneracy of the genetic code, encode the amino acid sequence of the sorghum elta-kafirin2 or sugar cane delta-prolamin2 proteins of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis, but which still-encode sorghum delta-kafirin2 or sugar cane delta-prolamin2 proteins. Generally, variants of a particular nucleotide sequence of the invention will have at least about 60%, 65%, 70%, 75%, 80%, 85%, 90%, 91%, 92%, 93%, 94%, 95%, 96%, 97%, 98%, or 99% or more sequence identity to that particular nucleotide sequence over a length of 20, 30, 50, or 100 nucleotides or less, as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess all or some of the activity of the native proteins of the invention as described herein. Such variants may result from, for example, genetic polymorphism or from human manipulation. Biologically active variants of the native sorghum delta-kafirin2 or sugarcane delta-prolamin2 proteins of the invention will have at least about 60%, 65%, 70%, 75%, 80%, 85%, 90%, 91%, 92%, 93%, 94%, 95%, 96%, 97%, 98%, or 99% or more sequence identity to the amino-acid sequence for the native protein over a length of 10, 30, 50, or 100 amino acid residues or less as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the sorghum delta-kafirin2 or sugarcane delta-prolamin2 proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be-found in the model of Dayhoff et al. (1978) Atlas of Protein Sequence and Structure (Natl. Biomed. Res. Found., Washington, D.C.), herein incorporated by reference.

Thus, the genes and nucleotide sequences of the invention include both the naturally occurring sequences as well as mutant forms. Likewise, the proteins of the invention encompass both naturally occurring variant proteins as well as variations and modified forms thereof. Such variants will continue to be biologically active as defined herein. Obviously, the mutations that will be made in the DNA encoding the variant must not place the sequence out of reading frame and preferably will not create complementary regions that could produce secondary mRNA structure. See EP Patent Application Publication No. 75,444.

Variant nucleotide sequences and proteins also encompass sequences and proteins derived from a mutagenic and recombinogenic procedure such as DNA shuffling. With such a procedure, one or more different kafirin or prolamin protein coding sequences can be manipulated to create a new kafirin or prolamin protein possessing the desired properties. In this-manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between coding sequences of the invention and other known gene coding sequences to obtain a new coding sequence for a protein with an improved property of interest. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention and known seed proteins can be used to isolate corresponding sequences from other plants. In this manner, methods such as PCR, hybridization, and the like can be used to identify such sequences based on their sequence homology to the sequence set forth herein. Sequences isolated based on their sequence identity to known abundant corn seed proteins and the entire sorghum delta-kafirin2 and sugarcane delta-prolamin2 sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on, for example, the sorghum delta-kafirin2 sequence of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire sorghum delta-kafirin2 or sugar cane delta-prolamin2 sequence disclosed herein, or one or more portions thereof, may be used as a probe capable of specifically hybridizing to corresponding seed protein sequences and messenger RNAs. To achieve specific hybridization under a variety of conditions, such probes include sequences that are unique among the seed protein sequences of the invention and are preferably at least about 40 nucleotides in length. Such probes may be used to amplify corresponding sequences from a chosen plant by PCR. This technique may be used to isolate additional coding sequences from a desired plant or as a diagnostic assay to determine the presence of coding sequences in a plant. Hybridization techniques-include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode polypeptides that function as a seed protein and which hybridize under stringent conditions to the sorghum delta-kafirin2 or sugarcane delta-prolamin2 protein sequence disclosed herein, or to fragments thereof, are encompassed by the present invention. Such sequences will be at least about 60%, 65%, 70%, 75%, 80%, 85%, 90%, 91%, 92%, 93%, 94%, 95%, 96%, 97%, 98%, or 99% or more homologous with the disclosed sequence. That is, the sequence identity of sequences may range, sharing at least about 60%, 65%, 70%, 75%, 80%, 85%, 90%, 91%, 92%, 93%, 94%, 95%, 96%, 97%, 98%, or 99% or more sequence identity.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program, aligned over the full length of the sequence. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 60%, 65%, 70%, 75%, 80%, 85%, 90%, 91%, 92%, 93%, 94%, 95%, 96%, 97%, 98%, or 99% or more sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, 65%, 70%, 75%, 80%, 85%, 90%, 91%, 92%, 93%, 94%, 95%, 96%, 97%, 98%, or 99% or more.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 60%, 65%, 70%, 75%, 80%, 85%, 90%, 91%, 92%, 93%, 94%, 95%, 96%, 97%, 98%, or 99% or more sequence identity to the reference sequence over a specified comparison window. Alignment can be conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Peptides that are “substantially similar” comprise a sequence with at least 60%, 65%, 70%, 75%, 80%, 85%, 90%, 91%, 92%, 93%, 94%, 95%, 96%, 97%, 98%, or 99% or more sequence identity or sequence similarity to the reference sequence over a specified comparison window. In this case residue positions that are not identical instead differ by conservative amino acid changes.

Analysis of cDNA sequence from a sorghum developing seed cDNA library has resulted in identification of a nucleotide sequence, SEQ ID NO: 1 and the encoded amino acid sequence, SEQ ID NO: 2, which exemplifies a sulfur-amino acid rich polypeptide. The amino acid sequence of SEQ ID NO: 2 contains 191 residues with a molecular weight predicted of 21 kD, and has a distant homology to delta zeins from corn (about 15%). The amino acid shares a 25% global identity and higher identity in local alignment, along with structural similarly at the N- and C-terminal domains to a previously described delta-kafirin1, described at GenBank Accession No. AAW32936 by Izquierdo, L. Y. and Godwin, I. D. (2004).

Based on this similarity, the name “delta-kafirin2” was chosen for SEQ ID NO: 2. This 191 amino acid sorghum delta kafirn2 polypeptide (SEQ ID NO: 2) is a pre-propolypeptide containing at the N-terminus a predicted 26-amino acid long Endoplasmic Reticulum (ER) targeting sequence (signal peptide). The signal peptide is proteolytically removed from the pro-peptide upon targeting into the ER and this processing step results in a 165-amino acid mature delta-kafirin2 polypeptide with a calculated molecular weight of ˜18 KD that is the primary form of its accumulation in sorghum seed.

Further analysis of ESTs from sugar cane in public databases (GenBank) revealed a highly similar gene in sugarcane encoding a protein, having 90% identity at the amino acid level to delta-kafirn2. The ESTs were aligned and assembled to produce the predicted nucleotide sequence of SEQ ID NO: 3, and the amino acid shown at SEQ ID NO: 4, identified here as sugarcane delta-prolamin2. It is 178 amino acid residues long with a predicted molecular weight of 19.5 kD.

Both the sorghum kafirins and the sugarcane prolamins are rich in sulfur amio acids, having about 27% sulfur amino acids by frequency; and are also rich in threonine (about 6% by frequency). The sorghum delta-kafirin2 has three lysine residues and the sugar cane delta-prolamin2 has two lysine residues.

The amino acid sequences of delta-kafirin2 and delta-prolamin2 can be aligned to the amino acid sequence of delta-kafirin1 and the 10 kD delta-zein using ClustalW software and this alignment shows the signature arrangement of conserved cysteine residues of polypeptides belonging to the 2S albumin superfamily of plant proteins (pfam domain PF00234). The sorghum delta-kafirin2 and the sugarcane delta-prolamin2 proteins are deposited in maturing seed and function as storage proteins, i.e. the amino acids contained in these proteins are mobilized during seed germination and provide nutrients to the growing seedling.

By “disulfide status” is intended the portion of cysteine residues within a protein that participate in disulfide bonds or disulfide bridges. Such disulfide bonds can be formed between the sulfur of a first cysteine residue and the sulfur of a second cysteine residue. It is recognized that such first and second cysteine residues can occur as part of a single polypeptide chain, or alternatively, can occur on separate polypeptide chains referred to herein as “inter-molecular disulfide bonds”. When “disulfide status” is used in reference to a seed or part thereof, the “disulfide status” of such a seed or part thereof is the total disulfide status of the proteins therein.

Disulfide-rich protein fractions in grain has been implicated as a major determinant of poor amino acid content which contributes to its low nutrient content. In addition, a high disulfide status it can also be a significant contributor to the wet-milling properties of grain. For example, in the wet-milling process, the higher the number of disulfide bonds, the greater the requirement for chemical reductants to break these bonds and to maximize the release of starch granules. It is believed that extensive disulfide bonding negatively impacts the process of wet-milling.

Intermolecular disulfide bridges are also important for the formation and maintenance of protein bodies. These protein bodies contribute to the physical properties of the grain that also affect the wet-milling process. In the wet-milling process, chemical reductants are required to break protein disulfide bonds to maximize starch yield and quality (Hoseney, R. C. (1994), Principles of Cereal Science and Tech., (Ed.2)). The use in wet mills of odorous chemical such as sulfur dioxide and bisulfite requires extensive precautions and poses significant environmental problems.

Similar to that described for a decrease in the number of disulfide bonds, a decrease in the number of protein bodies can also be expected to improve the efficiency of the wet-milling process. Seed proteins interact during formation of protein bodies (through intermolecular disulfide bonds and hydropobic interactions), and these interactions are important for the formation of proteolytically stable complexes. Though not limited by any theory of action, a decrease in the expression of two or three seed protein genes can be expected to have an additive effect on the reduction of protein bodies resulting in a corresponding improvement in wet-milling properties.

The wet-milling properties of the grain of the present invention can be analyzed using a small-scale simulated wet-milling process incorporating or leaving out a reducing agent (bisulfite) in the steep water as used by Eckhoff et al., (1996, Cereal Chem. 73:54-57).

In addition to the positive impact that reducing agents have on the release of starch granules in the wet-milling process, it has also been shown that reducing agents can increase the dry matter digestibility of sorghum and corn and, thus, improve their feed properties. This result is supported by the results of data from in vitro digestibility assays described in the present invention that demonstrate that reducing agents increase dry matter digestibility or energy availability. See also: Hamaker, B. R., et al., 1987, Improving the in vitro protein digestibility of sorghum with reducing agents, Proc. Natl. Acad. Sci. USA 84:626-628.

The “energy value”, or “caloric value” of a feed or food, which is determined by energy density or gross energy (GE) content and by energy availability, is also termed “metabolizable energy (ME) content.” (see Wiseman, J., and Cole, D. J. A., (1987), Animal Production 45(1):117-122)

As used herein, “energy availability” means the degree to which energy-rendering nutrients are available to the animal, often referred to as energy conversion (ratio of metabolizable energy content to gross energy content). One way energy availability may be determined is with in vivo balance trials, in which excreta are collected by standard methodology (e.g., Sibbald, I. R., Poultry Science, 58(5):1325-29 (1979); McNab and Blair, British Poultry Science 29(4):697-708 (1988)). Energy availability is largely determined by food or feed digestibility in the gastrointestinal tract, although other factors such as absorption and metabolic utilization also influence energy availability.

“Digestibility” is defined herein as the fraction of the feed or food that is not excreted in feces or urine. Digestibility is a component of energy availability. It can be further defined as digestibility of specific constituents (such as carbohydrates or protein) by determining the concentration of these constituents in the foodstuff and in the excreta. Digestibility can be estimated using in vitro assays, which is routinely done to screen large numbers of different food ingredients and plant varieties. In vitro techniques, including assays with rumen inocula and/or enzymes for ruminant livestock (e.g. Pell and Schofield, Journal of Dairy Science 76(4):1063-1073 (1993)) and various combinations of enzymes for monogastric animals reviewed in Boisen and Eggum, Nutrition Research Reviews 4:141-162 (1991) are also useful techniques for screening transgenic materials for which only limited sample is available.

The enzyme digestible dry matter (EDDM) assay used in these experiments as an indicator of in vivo digestibility is known in the art and can be performed according to the methods described in Boisen and Fernandez (1997) Animal Feed Science and Technology 68:277-286, and Boisen and Fernandez (1995) Animal Feed Science and Technology 51:29-43; which are herein incorporated in their entirety by reference. The actual in vitro method used for determining EDDM in this patent application is a modified version of the above protocol as described in Example 2. These data indicate that reducing the number of disulfide bonds in the seed of sorghum and corn can increase the dry matter digestibility of grain from these crops while retaining a “normal” i.e.: vitreous phenotype. It is also likely that a decrease in the disulfide-status of other grains would have a similar positive effect on their digestibility properties.

While seed with extensive disulfide bonding exhibits poor wet-milling properties and decreased dry matter digestibility, a high disulfide-status has also been correlated with increased seed hardness and improved dry-milling properties. Assays for seed hardness are well known in the art and include such methods as those used in the present invention, described in Pomeranz et al. (1985) Cereal Chemistry 62:108-112; herein incorporated in its entirety by reference.

The “nutritional value” of a feed or food is defined as the ability of that feed or food to provide nutrients to animals or humans. The nutritional value is determined by three factors: concentration of nutrients (protein & amino acids, energy, minerals, vitamins, etc.), their physiological availability during the processes of digestion, absorption and metabolism, and the absence (or presence) of anti-nutritional compounds.

It has been demonstrated that proteolytic digestion of the alcohol-soluble seed protein fraction (prolamins) from wheat, barley, oats, and rye is known to give rise to anti-nutritional peptides able to adversely affect the intestinal mucosa of coeliac patients (Silano and Vincenzi (1999) Nahrung 43:175-184). Furthermore, the alpha-, beta-, and gamma-gliadins present in the prolamin-like protein fraction of wheat are capable of inducing coeliac disease (Friis et al. (1994) Clin. Chim. Acta. 231:173-183). The alpha-gliadin and gamma-gliadin from wheat have also been identified as major allergens (Maruyama et al. (1998) Eur. J. Biochem. 256:604. For these reasons the methods of the present invention are also directed to the elimination or the reduction of the levels of at least one seed protein in wheat, barley, oats, or rye to produce a grain with eliminated or reduced anti-nutritional or allergenic properties.

The compositions and methods of the invention are useful for modulating the levels of at least one seed protein in seeds. By “modulate” is defined herein as an increase or decrease in the level of a seed protein within seed of a genetically altered plant relative to the level of that protein in seed from the corresponding wild-type plant (i.e., a plant not genetically altered in accordance with the methods of the present invention).

The terms “inhibit”, “inhibition”, “inhibiting”, “reduced”, “reduction” and the like as used herein refer to any decrease in the expression or function of a target gene product, including any relative decrement in expression or function up to and including complete abrogation of expression or function of the target gene product. The term “expression” as used herein in the context of a gene product refers to the biosynthesis of that gene product, including the transcription and/or translation of the gene product. Inhibition of expression or function of a target gene product (i.e., a gene product of interest) can be in the context of a comparison between any two plants, for example, expression or function of a target gene product in a genetically altered plant versus the expression or function of that target gene product in a corresponding wild-type plant. Alternatively, inhibition of expression or function of the target gene product can be in the context of a comparison between plant cells, organelles, organs, tissues, or plant parts within the same plant or between plants, and includes comparisons between developmental or temporal stages within the same plant or between plants. Any method or composition that down-regulates expression of a target gene product, either at the level of transcription or translation, or down-regulates functional activity of the target gene product can be used to achieve inhibition of expression or function of the target gene product.

The term “inhibitory sequence” encompasses any polynucleotide or polypeptide sequence that is capable of inhibiting the expression of a target gene product, for example, at the level of transcription or translation, or which is capable of inhibiting the function of a target gene product. Examples of inhibitory sequences include, but are not limited to, full-length polynucleotide or polypeptide sequences, truncated polynucleotide or polypeptide sequences, fragments of polynucleotide or polypeptide sequences, variants of polynucleotide or polypeptide sequences, sense-oriented nucleotide sequences, antisense-oriented nucleotide sequences, the complement of a sense- or antisense-oriented nucleotide sequence, inverted regions of nucleotide sequences, hairpins of nucleotide sequences, double-stranded nucleotide sequences, single-stranded nucleotide sequences, combinations thereof, and the like. The term “polynucleotide sequence” includes sequences of RNA, DNA, chemically modified nucleic acids, nucleic acid analogs, combinations thereof, and the like.

Inhibitory sequences are designated herein by the name of the target gene product. Thus, for example, a “sorghum delta-kafirin2” would refer to an inhibitory sequence that is capable of inhibiting the expression of sorghum delta-kafirin2, for example, at the level of transcription and/or translation, or which is capable of inhibiting the function of sorghum delta-kafirin2. When the phrase “capable of inhibiting” is used in the context of a polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded (ds) RNA polynucleotides), or encodes an inhibitory polypeptide (i.e., a polypeptide that inhibits expression or function of the target gene product), following its transcription (for example, in the case of an inhibitory sequence encoding a hairpin RNA, miRNA, or dsRNA polynucleotide) or its transcription and translation (in the case of an inhibitory sequence encoding an inhibitory polypeptide), the transcribed or translated product, respectively, exerts the inhibitory effect on the target gene product (i.e., inhibits expression or function of the target gene product).

Conversely, the terms “increase,” “increased,” and “increasing” in the context of the methods of the present invention refer to any increase in the expression or function of a gene product, including any relative increment in expression or function. As with inhibition, increases in the expression or function of a gene product of interest (i.e., a target gene product) can be in the context of a comparison between any two plants, for example, expression or function of a target gene product in a genetically altered plant versus the expression or function of that target gene product in a corresponding wild-type plant. Alternatively, increases in the expression or function of the target gene product can be in the context of a comparison between plant cells, organelles, organs, tissues, or plant parts within the same plant or between plants, and includes comparisons between developmental or temporal stages within the same plant or between plants. Any method or composition that up-regulates expression of a target gene product, either at the level of transcription or translation, or up-regulates functional activity of the target gene product can be used to achieve increased expression or function of the target gene product.

In one embodiment, methods are particularly directed to reducing the level of seed proteins, such as, but not limited to, sorghum delta-kafirin2 and sugarcane prolamin2 proteins to improve the nutritional value and industrial use of grain. In another embodiment is reduction of the cysteine-rich amino acid seed proteins of the sorghum delta-kafirin2, and the sugar cane delta-prolamin 2. Other embodiments of the invention include methods directed to screening for particular plant phenotypes based on antibodies specific for the polypeptides of the invention, or using SNP's of the nucleotide sequences of the invention.

Reduction of the level of the sorghum kafirins or sugar cane prolamins in plant seed can be used to improve the nutritional value and industrial use of such grain. The methods of the invention can be useful for producing grain that is more rapidly and extensively digested than grain with normal/wild-type prolamin or kafirin protein levels.

Reduction of the level of kafirin proteins in plant seed, and homologous sugar cane prolamins, can be used to improve the nutritional value and industrial use of such grain. The methods of the invention are also useful for producing grain that is more rapidly and extensively digested than grain with normal gamma-zein protein levels.

Inhibition of kafirin genes can be used to increase the nutritional value of seed, particularly by increasing the energy availability of seed. Reduction in the kafirin levels in such seed can be at least about 20%, 30%, 40%, 50%, 60%, 70%, 80%, 90% and up to 100%. Energy availability can be improved by at least 3%, 6%, 9%, 12%, 15%, 20% and greater.

Methods of the invention are also directed to the reduction or elimination of the expression of one or more specific prolamin-like proteins in the grain of wheat, barley, oats, and rye that are known to give rise to anti-nutritional peptides. These proteins include, but are not limited to, the alpha-, beta-, and gamma-gliadins of wheat. Grain and grain products possessing reduced levels of these proteins would not possess such negative characteristics as inducing coeliac disease or stimulating an allergic response.

It is noted that modifications made to the grain by the present invention typically do not compromise grain handling properties with respect to mechanical damage: taking into account that grain handling procedures are adapted to specific properties of the modified grain. Mechanical damage to grain is a well-described phenomenon (e.g., McKenzie, B. A., Am Soc Ag Engineers (No: 85-3510): 10 pp, 1985) that contributes to dust in elevators and livestock facilities, and which may increase susceptibility to pests. Grain damage can be quantified and assessed by objective measures (e.g., Gregory, J. M., et al., Am Soc. Ag. Engineers (no. 91-1608): 11pp, 1991) such as kernel density and test weight. See also: McKenzie, B. A. 1985, supra.

Methods of the invention can be utilized to alter the level of any seed protein found within a particular plant species, including but not limited to, the delta-kafirin2 of sorghum, the legumin 1 and other seed proteins of maize, rice and sorghum, the delta-prolamin2 of sugarcane, and the alpha-, beta-, and gamma-gliadins of wheat, barley, rye, and oats.

In many instances the nucleotide sequences for use in the methods of the present invention, are provided in transcriptional units with for transcription in the plant of interest. A transcriptional unit is comprised generally of a promoter and a nucleotide sequence operably linked in the 3′ direction of the promoter, optionally with a terminator.

By “operably linked” is intended a functional linkage between a promoter and a second sequence, wherein the promoter sequence initiates and mediates transcription of the DNA sequence corresponding to the second sequence. The expression cassette will include 5′ and 3′ regulatory sequences operably linked to at least one of the sequences of the invention.

Generally, in the context of an over expression cassette, operably linked means that the nucleotide sequences being linked are contiguous and, where necessary to join two or more protein coding regions, contiguous and in the same reading frame. In the case where an expression cassette contains two or more protein coding regions joined in a contiguous manner in the same reading frame, the encoded polypeptide is herein defined as a “heterologous polypeptide” or a “chimeric polypeptide” or a “fusion polypeptide”. The cassette may additionally contain at least one additional coding sequence to be co-transformed into the organism. Alternatively, the additional coding sequence(s) can be provided on multiple expression cassettes.

The methods of transgenic expression can be used to increase the level of at least one seed protein in grain. The methods of transgenic expression comprise transforming a plant cell with at least one expression cassette comprising a promoter that drives expression in the plant operably linked to at least one nucleotide sequence encoding a seed protein. Methods for expressing transgenic genes in plants are well known in the art.

In other instances the nucleotide sequences for use in the methods of the invention are provided in transcriptional units as co-supression cassettes for transcription in the plant of interest. Transcription units can contain coding and/or non-coding regions of the genes of interest. Additionally, transcription units can contain promoter sequences with or without coding or non-coding regions. The co-suppression cassette may include 5′ (but not necessarily 3′) regulatory sequences, operably linked to at least one of the sequences of the invention. Co-supression cassettes used in the methods of the invention can comprise sequences of the invention in so-called “inverted repeat” structures. The cassette may additionally contain a second copy of the fragment in opposite direction to form an inverted repeat structure: opposing arms of the structure may or may not be interrupted by any nucleotide sequence related or unrelated to the nucleotide sequences of the invention. (see Fiers et al. U.S. Pat. No. 6,506,559). The transcriptional units are linked to be co-transformed into the organism. Alternatively, additional transcriptional units can be provided on multiple over-expression and co-suppression cassettes.

The methods of transgenic co-suppression can be used to reduce or eliminate the level of at least one seed protein in grain. One method of transgenic co-suppression comprise transforming a plant cell with at least one transcriptional unit containing an expression cassette comprising a promoter that drives transcription in the plant operably linked to at least one nucleotide sequence transcript in the sense orientation encoding at least a portion of the seed protein of interest. Methods for suppressing gene expression in plants using nucleotide sequences in the sense orientation are known in the art. The methods generally involve transforming plants with a DNA construct comprising a promoter that drives transcription in a plant operably linked to at least a portion of a nucleotide sequence that corresponds to the transcript of the endogenous gene. Typically, such a nucleotide sequence has substantial sequence identity to the sequence of the transcript of the endogenous gene, at least about 65%, 70%, 75%, 80%, 85%, 90%, 91%, 92%, 93%, 94%, 95%, 96%, 97%, 98%, or 99% or more sequence identity over the entire length of the sequence. Furthermore, portions, rather than the entire nucleotide sequence, of the polynucleotides may be used to disrupt the expression of the target gene product. Generally, sequences of at least 10, 15, 20, 21, 22, 23, 24, 25, 26, 27, 28, 29, 30, 35, 40, 45, 50, 55, 60, 65, 70, 75, 80, 85, 90, 95, 100, 200 nucleotides, or greater may be used. See U.S. Pat. Nos. 5,283,184 and 5,034,323; herein incorporated by reference.

The endogenous gene targeted for co-suppression may be a gene encoding any seed protein that accumulates as a seed protein in the plant species of interest, including, but not limited to, the seed genes noted above. For example, where the endogenous gene targeted for co-suppression is the sorghum delta-kafirin2 gene disclosed herein, co-suppression is achieved using an expression cassette comprising the sorghum delta-kafirin2 gene sequence, or variant or fragment thereof.

Additional methods of co-suppression are known in the art and can be similarly applied to the instant invention. These methods involve the silencing of a targeted gene by spliced hairpin RNA's and similar methods also called RNA interference and promoter silencing (see Smith et al. (2000) Nature 407:319-320, Waterhouse and Helliwell (2003) Nat. Rev. Genet. 4:29-38; Waterhouse et al. (1998) Proc. Natl. Acad. Sci. USA 95:13959-13964; Chuang and Meyerowitz (2000) Proc. Natl. Acad. Sci. USA 97:4985-4990; Stoutjesdijk et al. (2002) Plant Phystiol. 129:1723-1731; and Patent Application WO 99/53050; WO 99/49029; WO 99/61631; WO 00/49035 and U.S. Pat. No. 6,506,559, each of which is herein incorporated by reference). For the purpose of this invention the term “co-suppression” is used to collectively designate gene silencing methods based on mechanisms involving the expression of sense RNA molecules, aberrant RNA molecules, dsRNA molecules, micro RNA molecules and the like.

The expression cassette for co-suppression may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, International Publication No. WO 02/00904, herein incorporated by reference.

In other embodiments of the invention, inhibition of the expression of a protein of interest may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). miRNA molecules are highly efficient at inhibiting the expression of endogenous genes, and the RNA interference they induce is inherited by subsequent generations of plants.

In one embodiment, the polynucleotide to be introduced into the plant comprises an inhibitory sequence that encodes a zinc finger protein that binds to a gene encoding a protein of the invention resulting in reduced expression of the gene. In particular embodiments, the zinc finger protein binds to a regulatory region of a zein gene, a legumin gene or a globulin gene. In other embodiments, the zinc finger protein binds to a messenger RNA encoding a seed protein and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. Patent Publication No. 2003/0037355; each of which is herein incorporated by reference.

Methods for antisense suppression can be used to reduce or eliminate the level of at least one seed protein in grain. The methods of antisense suppression comprise transforming a plant cell with at least one expression cassette comprising a promoter that drives expression in the plant cell operably linked to at least one nucleotide sequence that is antisense to a nucleotide sequence transcript of such a gamma-zein gene. By “antisense suppression” is intended the use of nucleotide sequences that are antisense to nucleotide sequence transcripts of endogenous plant genes to suppress the expression of those genes in the plant.

Methods for suppressing gene expression in plants using nucleotide sequences in the antisense orientation are known in the art. The methods generally involve transforming plants with a DNA construct comprising a promoter that drives expression in a plant operably linked to at least a portion of a nucleotide sequence that is antisense to the transcript of the endogenous gene. Antisense nucleotides are constructed to hybridize with the corresponding mRNA. Modifications of the antisense sequences may be made as long as the sequences hybridize to and interfere with expression of the corresponding mRNA. In this manner, antisense constructions having at least about 70%, 75%, 80%, 85%, 90%, 91%, 92%, 93%, 94%, 95%, 96%, 97%, 98%, or 99% or more sequence identity to the corresponding antisense sequences may be used. Furthermore, portions, rather than the entire nucleotide sequence, of the antisense nucleotides may be used to disrupt the expression of the target gene. Generally, sequences of at least 10 nucleotides, 50 nucleotides, 100 nucleotides, 200 nucleotides, or greater may be used.

Methods for transposon tagging can be used to reduce or eliminate the level of at least one seed protein in grain. The methods of transposon tagging comprise insertion of a transposon within an endogenous plant seed gene to reduce or eliminate expression of the seed protein.

Methods for transposon tagging of specific genes in plants are well known in the art (see for example, Maes et al. (1999) Trends Plant Sci. 4:90-96; Dharmapuri and Sonti (1999) FEMS Microbiol. Lett. 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nuc. Acids Res. 28:94-96; Fitzmaurice et al. (1999) Genetics 153:1919-1928). In addition, the TUSC process for selecting Mu-insertions in selected genes has been described (Bensen et al. (1995) Plant Cell 7:75-84; Mena et al. (1996) Science 274:1537-1540; U.S. Pat. No. 5,962,764, which is herein incorporated by reference).

Other methods for inhibiting or eliminating the expression of endogenous genes are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted (for examples of these methods see Ohshima et al. (1998) Virology 243:472-481; Okubara et al. (1994) Genetics 137:867-874; Quesada et al. (2000) Genetics 154:421-436. In addition, a fast and automatable method for screening for chemically induced mutations, TILLING, (Targeting Induced Local Lesions In Genomes), using a denaturing HPLC or selective endonuclease digestion of selected PCR products is also applicable to the instant invention (see McCallum et al. (2000) Nat. Biotechnol. 18:455-457).

Mutation breeding is another of many methods that could be used to introduce new traits into an elite line. Mutations that occur spontaneously or are artificially induced can be useful sources of variability for a plant breeder. The goal of induced mutagenesis is to increase the rate of mutation for a desired characteristic. Mutation rates can be increased b many different means including: temperature; long-term seed storage; tissue culture conditions; radiation such as X-rays, Gamma rays (e.g., Cobalt 60 or Cesium 137), neutrons, (product of nuclear fission by Uranium 235 in an atomic reactor, Beta radiation (emitted from radioisotopes such as P32, or C14), or ultraviolet radiation (preferably from 2500 to 2900 nm); or chemical mutagens such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis, the trait may then be incorporated into existing germplasm by traditional breeding techniques, such as backcrossing. Details of mutation breeding can be found in “Principals of Cultivar Development” Fehr, 1993 (Macmillan Publishing Company), the disclosures of which are incorporated herein by reference. In addition, mutations created in other lines may be used to produce a backcross conversion of elite lines that comprise such mutations.

Other methods for inhibiting or eliminating the expression of genes include the transgenic application of transcription factors (Pabo, C. O., et al. (2001) Annu Rev Biochem 70, 313-40; and Reynolds, L., et al (2003), Proc Natl Acad Sci USA 100, 1615-20.), and homologous recombination methods for gene targeting (see U.S. Pat. No. 6,187,994).

Similarly, it is possible to eliminate the expression of a single gene by replacing its coding sequence with the coding sequence of a second gene using homologous recombination technologies (see Bolon, B. Basic Clin. Pharmacol. Toxicol. 95:4-12, 154-61 (2004); Matsuda and Alba, A., Methods Mol. Bio. 259:379-90 (2004); Forlino, et al., J. Biol. Chem. 274:53, 37923-30 (1999)). For example, by using the knock-out/knock-in technology, the coding sequence of the 27 kD gamma-zein protein can be replaced by the coding sequence of the 18 kD alpha-globulin resulting in suppression of 27 kD gamma-zein protein expression and in over-expression of the alpha-globulin protein.

In some embodiments, the polynucleotide expressed by the expression cassette of the invention is catalytic RNA or has ribozyme activity specific for the messenger RNA of a protein of interest. Thus, the polynucleotide causes the degradation of the endogenous messenger RNA, resulting in reduced expression of one or more proteins. This method is described, for example, in U.S. Pat. No. 4,987,071, herein incorporated by reference.

In some embodiments of the invention, the polynucleotide comprises an inhibitory sequence that encodes an antibody that binds to at least one isoform of a seed protein, and reduces the level of the seed protein. In another embodiment, the binding of the antibody results in increased turnover of the antibody-antigen complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21:35-36, incorporated herein by reference.

Methods of biosynthetic competition with other high-sulfur-containing proteins are used to reduce the levels of at least one seed protein in plant seed. The methods of biosynthetic competition comprise transforming plant cells with at least one expression cassette comprising a promoter that drives expression in the plant cell operably linked to at least one nucleotide sequence encoding a protein selected from the group consisting of delta-zeins, hordothionin 12, and other naturally occurring or engineered high-sulfur-containing proteins. In some cases the competing protein may possess a high lysine content in addition to a high sulfur content to further increase the nutritional value of the grain.

Biosynthetic competition of seed proteins with other sulfur-rich proteins occurs naturally. This natural process can be manipulated to reduce the levels of certain seed proteins, because the synthesis of some seed proteins is transcriptionally and/or translationally controlled by the nitrogen and/or sulfur supply in the developing seed. The expression of recombinant polypeptides, including the ectopic (transgenic) expression of seed proteins or other high-sulfur-, high-nitrogen-containing proteins, can have a substantial impact on intracellular nitrogen and sulfur pools. Thus, the expression of these proteins can result in suppression of the expression of other seed proteins such as, for example, the high-sulfur containing gamma-zein proteins.

Plant transformants containing a desired genetic modification as a result of any of the above described methods resulting in increased, decreased or eliminated expression of the seed protein of the invention can be selected by various methods known in the art. These methods include, but are not limited to, methods such as SDS-PAGE analysis, immunoblotting using antibodies which bind to the seed protein of interest, single nucleotide polymorphism (SNP) analysis, or assaying for the products of a reporter or marker gene, and the like.

Another embodiment is directed to the screening of transgenic plants for specific phenotypic traits conferred by the expression, or lack thereof, of known seed proteins and polypeptides of the invention. The specific phenotypic traits for which this method finds use include, but are not limited to, all of those traits listed herein. Crop lines can be screened for a particular phenotypic trait conferred by the presence or absence of known seed proteins using an antibody that binds selectively to one of these polypeptides. In this method, tissue from the maize line of interest is contacted with an antibody that selectively binds the seed-protein polypeptide for which the screen is designed. The development and use of antibodies for the detection of known seed proteins is described in Woo, et al, et seq. The amount of antibody binding is then quantified and is a measure of the amount of the seed-protein polypeptide present in the crop line. Methods of quantifying polypeptides by immunodetection in this manner are well known in the art. Such methodology can likewise be applied to screening sorghum plants using the sorghum delta-kafirin2 and sugar cane delta-prolamin 2 proteins of the invention.

In the practice of certain specific embodiments of the present invention, a plant is genetically altered to have a suppressed or increased level of one or more seed proteins in seed and/or to ectopically express one or more seed or other high-sulfur, high-lysine-containing protein. Those of ordinary skill in the art realize that this can be accomplished in any one of a number of ways. For example, each of the respective coding sequences for such proteins can be operably linked to a promoter and then joined together in a single continuous fragment of DNA comprising a multigenic expression cassette. Such a multigenic expression cassette can be used to transform a plant to produce the desired outcome utilizing any of the methods of the invention including sense and antisense suppression and biosynthetic competition. Alternatively, separate plants can be transformed with expression cassettes containing one of the desired set of coding sequences. Transgenic plants resulting from any or a combination of methods including any method to modulate protein levels, can be selected by standard methods available in the art. These methods include, but are not limited to, methods such as immunoblotting using antibodies which bind to the proteins of interest, SNP analysis, or assaying for the products of a reporter or marker gene, and the like. Then, all of the desired coding sequences and/or transposon tagged sequences can be brought together into a single plant through one or more rounds of cross pollination utilizing the previously selected transformed plants as parents.

The nucleotide sequences for use in the methods of the present invention are provided in expression cassettes for transcription in the plant of interest. Such expression cassettes are provided with a plurality of restriction sites for insertion of the sorghum delta-kafirin2 or sugarcane delta-prolamin2 or any other sequence of the present invention to be placed under the transcriptional regulation of the regulatory regions. The expression cassettes may additionally contain selectable marker genes.

The expression cassette can include in the 5′-3′ direction of transcription, a transcriptional and translational initiation region, any seed protein sequence of the invention, and optionally, a transcriptional and translational termination region functional in plants. The transcriptional initiation region, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a gene comprises a coding sequence operably linked to a transcription initiation region that is heterologous to the coding sequence. Alternatively, a gene comprises fragments of at least two independent transcripts that are linked in a single transcription unit.

While it may be preferable to express the sequences using heterologous promoters, the native promoter sequences may be used. Such constructs would alter expression levels of the proteins in the plant or plant cell. Thus, the phenotype of the plant or plant cell is altered. Alternatively, the promoter sequence may be used to alter expression. For example, the promoter (or fragments thereof) of sorghum delta-kafirin2 can modulate expression of the native sorghum delta-kafirin2 protein or other closely related proteins.

Use of a termination region is not necessary for proper transcription of plant genes but may be used as part of an expression construct. The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, for example, as in the case of engineered high-sulfur-containing proteins for the method of biosynthetic competition, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome, only that the plant or cell thereof is altered as a result of the introduction of the nucleotide construct into a cell. In one embodiment of the invention, the genome may be altered following the introduction of the nucleotide construct into a cell. For example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

A number of promoters can be used in the practice of the invention. The promoters can be selected based on the desired outcome. The nucleic acids can be combined with constitutive, tissue-preferred, or other promoters for expression in plants, more preferably a promoter functional during seed development.

Such constitutive promoters include, for example, the core promoter of the Rsyn7 promoter and other constitutive promoters disclosed in WO 99/43838; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); PEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced protein expression within a particular plant tissue. Tissue-preferred promoters include, but are not limited to: Yamamoto et al. (1997) Plant J. 12(2)255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kD zein); and milps (myo-inositol-1-phosphate synthase; see U.S. Pat. No. 6,225,529 herein incorporated by reference). The 27 kD gamma-zein is a preferred endosperm-specific promoter. Glb-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kD zein, 22 kD zein, 27 kD zein, 10 kD delta-zein, waxy, shrunken 1, shrunken 2, globulin 1, etc.

In certain embodiments the nucleic acid sequences of the present invention can be combined with any combination of polynucleotide sequences of interest or mutations in order to create plants with a desired phenotype. For example, the polynucleotides of the present invention can be combined with any other polynucleotides of the present invention, such as any combination of SEQ ID NOS: 1, 3, 5, or with other seed storage protein genes or variants or fragments thereof such as: zeins, fatty acid desaturases, lysine ketoglutarate, lec1, or Agp. The combinations generated can also include multiple copies of any one of the polynucleotides of interest. The polynucleotides or mutations of the present invention can also be combined with any other gene or combination of genes to produce plants with a variety of desired trait combinations including, but not limited to, traits desirable for animal feed such as high oil genes (e.g., U.S. Pat. No. 6,232,529); balanced amino acids (e.g. hordothionins (U.S. Pat. Nos. 5,990,389; 5,885,801; 5,885,802; 5,703,409 and 6,800,726); high lysine (Williamson et al. (1987) Eur. J. Biochem. 165:99-106; and WO 98/20122); and high methionine proteins (Pedersen et al. (1986) J. Biol. Chem. 261:6279; Kirihara et al. (1988) Gene 71:359; and Musumura et al. (1989) Plant Mol. Biol. 12: 123)); and thioredoxins (U.S. application Ser. No. 10/005,429, filed Dec. 3, 2001)), the disclosures of which are herein incorporated by reference. The polynucleotides of the present invention can also be combined with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser et al. (1986) Gene 48:109); lectins (Van Damme et al. (1994) Plant Mol. Biol. 24:825); fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)), the disclosures of which are herein incorporated by reference. One could also combine the polynucleotides or mutations of the present invention with polynucleotides providing agronomic traits such as male sterility (e.g., see U.S. Pat. No. 5,583,210), stalk strength, flowering time, or transformation technology traits such as cell cycle regulation or gene targeting (e.g. WO 99/61619; WO 00/17364; WO 99/25821), the disclosures of which are herein incorporated by reference.

These combinations can be created by any method including, but not limited to, cross breeding plants by any conventional or TopCross methodology, by homologous recombination, site specific recombination, or other genetic modification. If the traits are combined by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. Traits may also be combined by transformation and mutation by any known method.

Methods of the invention can be utilized to alter the level of at lease one seed protein in seed from any plant species of interest. Plants of particular interest include grain plants that provide seeds of interest including grain seeds such as corn, wheat, barley, rice, sorghum, rye, oats, etc. The present invention may be used for many plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), oats, cassaya, and barley.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include, but are not limited to: microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840; Cai et al., U.S. patent application Ser. No. 09/056,418), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al.

(1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that the protein of interest of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

The cells that have been transformed may be grown into plants in accordance with conventional ways, under plant forming conditions. See, for example, McCormick et al. (1986) Plant Cell Reports 5:81-84. These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In addition, the desired genetically altered trait can be bred into other plant lines possessing desirable agronomic characteristics using conventional breeding methods and/or top-cross technology. The top-cross method is taught in U.S. Pat. No. 5,704,160 herein incorporated in its entirety by reference.

Methods for cross pollinating plants are well known to those skilled in the art, and are generally accomplished by allowing the pollen of one plant, the pollen donor, to pollinate a flower of a second plant, the pollen recipient, and then allowing the fertilized eggs in the pollinated flower to mature into seeds. Progeny containing the entire complement of heterologous coding sequences of the two parental plants can be selected from all of the progeny by standard methods available in the art as described infra for selecting transformed plants. If necessary, the selected progeny can be used as either the pollen donor or pollen recipient in a subsequent cross pollination.

It has been shown that the response in digestibility to the treatment of grain with DTT is inversely related to the digestibility of untreated grain (Boisen and Eggum, Nutrition Research Reviews 4:141-162 (1991)).

Digestibility of immature grain (grain at late dough or silage maturity stage) is equally improved by pretreatment with reducing agents (DTT) as mature grain. The same can be expected for low gamma-zein corn as the effects of DTT pretreatment, and low gamma-zein corn, on digestibility are virtually the same. Improvements in digestibility of immature grain through the methods of the present invention can be extrapolated to improvements in digestibility of silage—about half of which consists of immature grain. The improvements in digestibility with DTT pretreatment is inversely related to the intrinsic digestibility of untreated grain. For this reason, corn lines of low intrinsic digestibility can be expected to be more amenable to genetic modification through the method of the invention than those of higher digestibility. This aspect of the invention enables those of skill in the art of breeding to make rapid advances in introgressing a low gamma-zein trait into the appropriate elite germplasm.

This invention allows for the improvement of grain properties such as increased digestibility/nutrient availability, nutritional value, silage quality, and efficiency of wet or dry milling in strains already possessing other desirable characteristics.

Table of Sequence ID Nos.

| SEQ | | Amino Acid/ |
|---|---|---|
| ID NO: | Gene Name | Nucleotide |
| 1 | S.b. delta-kafirin2 | Nucleotide |
| 2 | S.b. delta-kafirin2 | Amino Acid |
| 3 | S.o. delta-prolamin2 | Nucleotide |
| 4 | S.o. delta-prolamin2 | Amino Acid |
| 5 | S.b. delta-kafirin2 signal sequence | Amino Acid |
| 6 | sorghum LKR | Nucleotide |
| 7 | sorghum LKR | Amino Acid |
| 8 | a-kafirin B1 primer 1372 | Nucleotide |
| 9 | a-kafirin B1 primer 1373 | Nucleotide |
| 10 | a-kafirin A1 primer 1374 | Nucleotide |
| 11 | a-kafirin A1 primer 1375 | Nucleotide |
| 12 | a-kafirin B2 primer 1376 | Nucleotide |
| 13 | a-kafirin B2 primer 1377 | Nucleotide |
| 14 | d-kafirin 2 primer 1378 | Nucleotide |
| 15 | d-kafirin 2 primer 1379 | Nucleotide |
| 16 | g-kafirin 1 primer 1380 | Nucleotide |
| 17 | g-kafirin 1 primer 1381 | Nucleotide |
| 18 | g-kafirin 2 primer 1382 | Nucleotide |
| 19 | g-kafirin 2 primer 1383 | Nucleotide |
| 20 | Sorhum LKR primer 1402 | Nucleotide |
| 21 | Sorhum LKR primer 1403 | Nucleotide |
| 22 | d-kafirin 2 pre-pro ppt primer (forward) | Nucleotide |
| 23 | d-kafirin 2 pre-pro ppt primer (reverse) | Nucleotide |

### EXAMPLES

### Example 1

### In Vitro Enzyme Digestible Dry Matter (EDDM) Assay

Grain is ground in a micro Wiley Mill (Thomas Scientific, Swedesboro, N.J.) through a 1 mm screen; 0.5 g of ground sample is placed in a pre-weighed nylon bag (50 micron pore size) and heat sealed. Approximately 40 bags are placed in an incubation bottle with 2 L of 0.2M phosphate buffer (pH 2.0) containing pepsin (0.25 mg/ml). Samples are incubated in a Daisy II incubator (ANKOM Technology, Fairport, N.Y.) at 39° C. for 2 hours. After 2 hours, samples are placed in a mesh bag and washed for 2 minutes with cold water in a washer (Whirlpool) using delicate cycle. Samples are then transferred into 2 L of 0.2M phosphate buffer (pH 6.8) containing pancreatin (5.0 mg/ml) and incubated at 39° C. for 4 or 6 hours. Samples are washed for 2 minutes as described earlier. Samples are then dried overnight at 55° C. and weighed. The difference in sample weight before and after incubation is expressed as percentage of enzyme digestible dry matter digestibility (EDDM). EDDM data generated by in vitro digestibility assay could vary with genetic backgrounds, field conditions and locations in which the plants are grown. Hence the absolute EDDM values could vary for the same transgene with different genetic backgrounds, field conditions and locations in which they are grown.

### Example 2

### Preparation of Sorghum delta-Kafirin2 and Sugarcane delta-Prolamin2-Specific Antibodies

Standard methods for the production of antibodies are used such as those described in Harlow and Lane (1988) Antibodies: A Laboratory Manual (Cold Spring Harbor, N.Y.: Cold Spring Harbor Laboratory; incorporated herein in its entirety by reference. Specifically, antibodies for sorghum delta-kafirin2 and sugarcane delta-prolamin2 polypeptides are produced by injecting female New Zealand white rabbits (Bethyl Laboratory, Montgomery, Tex.) six times with homogenized polyacrylamide gel slices containing 100 micrograms of PAGE purified polypeptide. The kafirin and prolamin polypeptides are purified by sub-cloning into a pET28 vector resulting in an insert encoding a His-tag fusion of the polypeptides. The fusion proteins are expressed in E. coli BL21 (DE3) cells and purified from the lysate by Nickel chelation chromatography. The denatured purified fusion proteins are used for immunization.

Animals are then bled at two week intervals. The antibodies are further purified by affinity-chromatography with Affigel 15 (BioRad)-immobilized antigen as described by Harlow and Lane (1988) Antibodies: A Laboratory Manual, Cold Spring Harbor, N.Y. The affinity column is prepared with purified kafirin and prolamin protein essentially as recommended by BioRad®. Immune detection of antigens on PVDF blots is carried out following the protocol of Meyer et al. (1988) J. Cell. Biol. 107:163; incorporated herein in its entirety by reference, using the ECL kit from Amersham (Arlington Heights, Ill.).

### Example 3

### Agrobacterium-Mediated Transformation of Maize

For Agrobacterium-mediated transformation of maize, a nucleotide sequence of the present invention is operably linked to either the 27 kD gamma-zein promoter or the maize CZ19B1 promoter to generate a transcriptional unit and this unit is incorporated into a Ti plasmid vector for Agrobacterium-based transformation, and the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the nucleotide sequence of interest to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### Example 4

### Agrobacterium-Mediated Transformation of Sorghum

For Agrobacterium-mediated transformation of sorghum the method of Cai et al. can be employed (U.S. patent application Ser. No. 09/056,418), the contents of which are hereby incorporated by reference). This method can be employed with a nucleotide sequence of the present invention using the promoters described in Example 3 herein, or another suitable promoter.

### Example 5

### Transformation of Maize Embryos by Particle Bombardment

Immature maize embryos from greenhouse donor plants are bombarded with a plasmid containing the nucleotide sequence of the present invention operably linked to a selected promoter plus a plasmid containing the selectable marker gene PAT (Wohlleben et al. (1988) Gene 70:25-37) that confers resistance to the herbicide Bialaphos. Transformation is performed as follows.

### Preparation of Target Tissue

The ears are surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

### Preparation of DNA

A plasmid vector comprising the nucleotide sequence encoding a protein of the present invention operably linked to a promoter is made. This plasmid DNA plus plasmid DNA containing a PAT selectable marker is precipitated onto 1.1 μm (average diameter) tungsten pellets using a CaCl₂ precipitation procedure as follows:
100 μl prepared tungsten particles in water
10 μl (1 μg) DNA in Tris EDTA buffer (1 μg total)
100 μl 2.5 M CaCl₂
10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

### Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

### Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for the desired phenotypic trait.

### Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-1 H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-1 H₂O following adjustment to pH 5.8 with KOH); 3.0 g/l Gelrite (added after bringing to volume with D-1 H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-1 H₂O after adjusting to pH 5.6); 3.0 g/l Gelrite (added after bringing to volume with D-1 H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-1 H₂O), 0.1 g/1 myo-inositol, and 40.0 g/l sucrose (brought to volume with polished D-1 H₂O after adjusting pH to 5.6); and 6 g/l bacto-agar (added after bringing to volume with polished D-1 H₂O), sterilized and cooled to 60° C.

### Example 6

### Transformation of Rice Embryogenic Callus by Bombardment

Embryogenic callus cultures derived from the scutellum of germinating seeds serve as the source material for transformation experiments. This material is generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos is then transferred to CM media (N6 salts, Nitsch and Nitsch vitamins, 1 mg/1 2,4-D, Chu et al., 1985, Sci. Sinica 18:659-668). Callus cultures are maintained on CM by routine sub-culture at two week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman #541 paper placed on CM media. The plates with callus are incubated in the dark at 27-28 C for 3-5 days. Prior to bombardment, the filters with callus are transferred to CM supplemented with 0.25 M mannitol and 0.25 M sorbitol for 3 hr. in the dark. The petri dish lids are then left ajar for 20-45 minutes in a sterile hood to allow moisture on tissue to dissipate.

Circular plasmid DNA from two different plasmids one containing the selectable marker for rice transformation and one containing the nucleotide of the invention, are co-precipitated onto the surface of gold particles. To accomplish this, a total of 10 μg of DNA at a 2:1 ratio of trait:selectable marker DNAs is added to a 50 μl aliquot of gold particles resuspended at a concentration of 60 mg ml-1. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are then washed twice with 1 ml of absolute ethanol and then resuspended in 50 μl of absolute ethanol and sonicated (bath sonicator) for one second to disperse the gold particles. The gold suspension is incubated at −70 C for five minutes and sonicated (bath sonicator) if needed to disperse the particles. Six μl of the DNA-coated gold particles are then loaded onto mylar macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Five to seven plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% gelrite+50 ppm hyg B) for 2 weeks in the dark at 25° C. After 2 weeks the callus is transferred to RM2 media (MS salts, Nitsch and Nitsch vitamins, 3% sucrose, 0.4% gelrite+50 ppm hyg B) and placed under cool white light (˜40 μEm⁻²s⁻¹) with a 12 hr photoperiod at 25° C. and 30-40% humidity. After 2-4 weeks in the light, callus generally begins to organize, and form shoots. Shoots are removed from surrounding callus/media and gently transferred to RM3 media (½×MS salts, Nitsch and Nitsch vitamins, 1% sucrose+50 ppm hygromycin B) in phytatrays (Sigma Chemical Co., St. Louis, Mo.) and incubation is continued using the same conditions as described in the previous step.

Plants are transferred from RM3 to 4″ pots containing Metro mix 350 after 2-3 weeks, when sufficient root and shoot growth has occurred. Plants are grown using a 12 hr/12 hr light/dark cycle using ˜30/18° C. day/night temperature regimen.

### Example 7

### Isolation of Kafirin Sequences from Sorghum and Highly Homologous Sequences from Sugar Cane

A sorghum seed cDNA library analysis identified a sulfur-amino acid rich seed protein, having about 27% sulfur amino acids by frequency. The encoded protein had a high methionine (38 residues, 20% by frequency) cysteine content (14 residues, 7% by frequency) and was also rich in threonine (12 residues, about 6% by frequency. The cDNA was sequenced and is SEQ ID NO: 1; the amino acid is SEQ ID NO: 2. Based upon similarity to the previously described delta-kafirin1 sequence (See GenBank accession No. AAW32936, supra), it was named delta-kafirin2. The sequence has 25% global identity and higher local identity to the delta-kafirin1 sequence, and shows structural similarity to the sequences, particularly at conserved cysteine residues in the N-terminal domain (Cys 44 and Cys 48 of the pre-propolypeptide) and in C-terminal domain (Cys 145 and Cys 147 of the pre-propolypeptide).

The delta-kafirin2 protein of SEQ ID NO:2 has 191 amino acid residues and its predicted molecular weight is 21 kD. This prepropolypeptide contains at the N-terminus a predicted 26-amino acid long Endoplasmic Reticulum (ER) targeting sequence (signal peptide). The signal peptide is proteolytically removed from the pro-peptide upon targeting into the ER. This processing step results in a 165-amino acid mature delta-kafirin2 polypeptide with a calculated molecular weight of ˜18 KD: the primary form of its accumulation in sorghum seed. The putative signal sequence of the delta-kafirin 2 amino acid is set forth is SEQ ID NO: 5.

A gene encoding a highly similar protein in sugar cane, having about 90% identity, was identified by analyzing expressed sequence tags (ESTs) from a sugar cane seed cDNA libraries. The nucleotide sequence was obtained from alignment and assembly of the ESTs and produced the cDNA sequence of SEQ ID NO: 3. The amino acid is SEQ ID NO: 4 and was named sugarcane delta-prolamin 2. It has 178 amino acid residues and a predicted molecular weight of 19.5 kD.

Kafirin fragments for RNAi cassette construction were obtained by PCR amplification from kafirin cDNA clones and from sorghum genomic DNA. A sorghum cDNA library from developing endosperm (20 days after pollination) was constructed and EST sequences were obtained from 1000 randomly selected cDNA clones. The EST sequences were clustered into EST contigs and analyzed to determine the complete transcript sequences and the relative expression levels of kafirin genes. Based on this analysis, conserved regions of the most abundantly expressed kafirin genes were selected for PCR amplification.

As templates for PCR amplification, either cDNA clones for specific kafirin sequences or sorghum genomic DNA were used. Methods for amplifying DNA fragments by using gene-specific primers are well known to the art. Gene fragments from alpha-kafirins B1, B2, and A1, gamma-kafirins 1 and 2, delta-kafirin 2 and lysine-keto-glutarate reductase(LKR) were amplified using primers that added convenient restriction sites to each (SEQ. ID NOS: 8-21).

### Example 8

### Modulating Seed Proteins in Sorghum

Building of Vectors for Agrobacterium-Mediated Plant Transformation.

Following sequence confirmation, the seven PCR fragments described above were ligated together to form a chimeric fragment. Two copies of this fragment were then ligated to form a self-complimentary hairpin construct with the two arms of the hairpin separated by an intervening spliceable intron and the entire cassette under the transcriptional control of the endosperm-specific promoter from the 19 KD alpha zein B1 gene of maize (see U.S. Pat. No. 6,225,529; issued May 1, 2001). In other variations of this chimeric construct, fragments representing one or more of the—kafirin groups (e.g. the alpha kafirins) were omitted from the chimeric.

In other variations the chimeric self-complimentary hairpin constructs are inserted into endosperm-specific promoter cassette either comprising the zea mays 27 kD gamma zein promoter, or any other endosperm preferred promoter that are known to the art.

Plant transformation vector for the above-described chimeric gene suppression cassettes were constructed. This cassette was subsequently introduced into Agrobacterium tumefaciens (LBA4404) carrying the superbinary vector PHP10523 (Japan Tobacco) and the resulting cointegrate was used in Agrobacterium-mediated transformation. Each step of vector construction was performed by standard DNA assembly techniques (See, for example, Sambrook et al. (1989) Molecular Cloning: A Laboratory Manual (Cold Spring Harbor, Laboratory Press, Plainview, N.Y.; or Gelvin et al., Plant Molecular Biology Manual (1990); each incorporated herein in its entirety by reference.). In some cases, cassettes were mobilized into pSB11-derived T-DNA vectors using Gateway™ homologous recombination technology (Invitrogen). After completion, the region between the T-DNA borders of each vector was sequenced in its entirety using standard sequencing technology.

DNA fragments for biolistic transformation (PMI system). Gene cassettes for biolistic transformation were isolated as linear DNA fragments from source plasmids after restriction digestion. Purification of DNA fragments by agarose electrophoresis was carried out twice to minimize the risk of contamination with plasmid backbone fragments (notably bacterial antibotic markers).

### Transformation

Agrobacterium-mediated transformation of sorghum was performed by the method described in Example 4.

Biolistic transformation was done by co-bombarding minimal concentrations of linearized transgene fragments and the PMI selectable marker cassettes. This strategy has been successfully used to minimize DNA rearrangements in transgenic plants (Loc, et al., 2002; Breitler, et al., 2002) and reduces the risk of trait loss due to transgene silencing. The PMI system (see above) addresses concerns often associated with transgenic crops by avoiding herbicide resistance for selection.

For each vector or construct up to 200 independent events were initially generated. This number produced at least 5 efficacious, high quality T0 events per vector available after event sorting for the breeding program. All T0 plants were grown in the greenhouse and self-pollinated. A minimum of 20 T1 seeds per event were harvested. Typically self-pollinated panicles segregated for the transgenes 3:1 (transgenic vs. non-transgenic).

### Event Sorting and Molecular Analysis

Event analysis has three major components: 1) PCR for transgene presence, 2) amino acid analysis, digestibility, seed protein and micronutrient analysis for trait efficacy and 3) PCR for trait gene copy number, absence of vector backbone DNA, herbicide gene elimination, and Southern for rearrangement analysis for regulatory compliance and compatibility selection.

The trait efficacy of the events was primarily assessed by protein expression analysis (protein electrophoresis, immune blotting) and by seed composition analysis using single seed. Altered expression of alpha and gamma-kafirin genes can easiest be assayed in stained protein gels and suppression of the delta-kafirin2 and sorghum bicolor LKR genes can be easiest assayed by immuno blotting Zein-and LKR antibodies that cross-reacted with corresponding sorghum proteins were used to assay for suppression of these sorghum proteins; the suppression of delta-kafirin2 is assayed with a delta-kafirin2 specific antibody (described in Example 2). Per each transgenic sorghum panicle, six seed that individually analyzed showed altered kafirin expression (“transgenic”) and six seed that showed no alteration in kafirin expression (“non-transgenic”) were used to generate a pool of transgenic seed and a pool of non-transgenic seed. The two pools were ground to a flour analyzed by amino acid analysis (acid hydrolysis) using standard techniques. The lysine content of the transgenic seed pools increased by at least 50% (per dry weight) when compared to the lysine content of the non transgenic seed pool.

These analysis techniques performed on single seed are routine and are well known to those of skill in the art. Grain samples are further evaluated for grain quality characteristics (hardness, grain moisture, test weight, grain digestibility) and grain yield. The outcome of this analysis is the selection of efficacious events for breeding and field release.

High-copy number and rearranged events and events with integrated vector backbone will be eliminated because of regulatory concerns (T0 plants). Because of gene flow issues, only events that do not contain the herbicide marker gene after Agrobacterium-mediated transformation are selected for breeding. Typically at least 20% of the events segregate for the marker gene. Segregation and elimination of the marker gene are assayed by PCR of 50 segregating T2 plants.

### Example 9

### Expression Cassettes for Accumulation of Delta-Kafirin2 Protein in Soybean Seed

The coding sequence of the delta-kafirin2 pre-pro-polypeptide was PCR amplified from SEQ ID NO: 1 with primers shown in SEQ ID NOS: 22 and 23, and digested with SmaI and BglII restriction enzymes to generate a DNA fragment with SmaI and BglII compatible ends at the 5′ and 3′ ends of the amplified PCR product, respectively.

A plasmid containing the Glycinin 1 promoter (GY Pro, nucleotides 1-690 of GenBank Acc# X15121) followed by a SmaI site, a BglII site and the BD30 terminator (nucleotides 6673-6460 of GenBank Acc# AB013289) was digested with SmaI and BglII to open it between the promoter and the BD30 terminator and the above described PCR product was sub-cloned by ligating it between these two sites resulting in expression cassette of GY1 PRO::Kafirin::BD30. Methods for amplifying DNA fragments by using gene-specific primers and methods for subcloning such fragments into expression cassettes are well known to the art. The sequence of expression cassette thus obtained was confirmed in its entirety by standard DNA sequence analysis.

Variants of this expression cassette are constructed with the soybean alpha prime beta-conglycinin promoter (for seed preferred expression), the soybean Kunitz trypsin inhibitor promoter, the beta-phaseolin promoter, the Psl lectin promoter or another promoter with expression in the seed and at the desired level. Yet another embodiment may use other 3′UTR sequences like the soybean alpha-prime beta-conglycinin 3′ UTR or the 3′ UTR of the beta-phasesolin gene.

### Example 10

### Transformation of Soybean

Soybean embryos are transformed with the expression cassettes described. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos that produce secondary embryos are then excised and placed into a suitable liquid medium. After repeated selection for clusters of somatic embryos that multiplied as early, globular staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A DuPont Biolistic PDS1000/HE instrument (helium retrofit) can be used for these transformations.

A selectable marker gene which can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the RNA suppression molecule and or the polypeptide of interest and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds, and the supernatant removed. The DNA-coated particles are then washed once in 400 μL 70% ethanol and resuspended in 40 μL of anhydrous ethanol. The DNA/particle suspension can be sonicated three times for one second each. Five μl of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 11

### Analysis of Transgenic Soybean Seed

Seed of transgenic soybean plants are harvested and 12 individual seed are ground to meal. Aliquots of the meals are extracted with SDS PAGE sample buffer (1:40 weight/volume) and subjected to analysis of the seed protein profiles by SDS-PAGE and immuno blotting with the delta-kafirin2 antibody. Immuno-positive seed are designated “transgenic” and immuno-negative seed are designated “non-transgenic”. Pools are made from transgenic seed and non-transgenic seed from transgenic lines with seed showing high levels of delta-kafirin2 accumulation and subjected to amino acid analysis after performic acid oxidation. Seed pools with accumulated delta-kafirin2 show a 50% in crease in sulfur amino acid accumulation when compared to pools of non-transgenic seed from the same heterozygous soybean lines.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated nucleic acid molecule comprising a nucleotide sequence selected from the group consisting of:
(a) a nucleotide sequence set forth in SEQ ID NO: 1, SEQ ID NO: 3, or SEQ ID NO: 6;
(b) a nucleotide sequence encoding the amino acid sequence of SEQ ID NO: 2, SEQ ID NO: 4 or SEQ ID NO: 7;
(c) a nucleotide sequence having at least 80% identity to the nucleotide sequence set forth in SEQ ID NO: 1, at least 80% identity to the nucleotide sequence set forth in SEQ ID NO: 3, or at least 95% identity to the nucleotide sequence set forth in SEQ ID NO: 6; wherein the % sequence identity is based on the entire sequence and is determined by GAP version 10 analysis using default parameters;
(d) a nucleotide sequence encoding a polypeptide having at least 80% identity to the amino acid sequence of SEQ ID NO: 2, at least 80% identity the amino acid sequence of SEQ ID NO: 4, or at least 95% identity to the amino acid sequence of SEQ ID NO: 7; wherein the % sequence identity is based on the entire sequence and is determined by GAP version 10 analysis using default parameters;
(e) a functional fragment of the nucleotide sequence of (a), (b), (c), (d) or (e); and
(f) an antisense nucleotide sequence corresponding to a nucleotide sequence of (a), (b), (c), (d) or (e).

2. A transformed plant comprising at least one nucleic acid molecule of claim 1.

3. Transformed seed of the plant of claim 2.

4. The plant of claim 2, wherein the plant is sorghum.

5. The plant of claim 2, wherein the plant is soybean.

6. An expression cassette comprising any one of the nucleotide sequences of claim 1.

7. A genetically modified cereal plant having improved grain digestibility as compared to the corresponding unmodified plant, the improved digestibility being due to decreased level of expression of a nucleotide sequence of claim 1.

8. The plant of claim 7 wherein the genetic modification is due to a mutation of the nucleotide sequence.

9. The plant of claim 7 wherein the genetic modification is due to co-suppression, antisense suppression, or RNAi-mediated suppression of the nucleotide sequence.

10. Genetically modified seed of the plant of claim 7.

11. A method for making a genetically modified plant with grain having improved digestibility as compared the corresponding unmodified plant, the method comprising:
a) introducing into a plant cell an expression cassette with means for decreasing the level of expression of a nucleotide sequence comprising at least one of the nucleotide sequences of claim 1;
b) regenerating a genetically modified plant from the cell; and
c) selecting for a genetically modified plant with grain having improved digestibility.

12. The method of claim 11 further comprising obtaining genetically modified progeny plants of one or more generations with improved digestibility.

13. The method of claim 11 wherein the means for decrease is co-suppression, antisense suppression, or RNAi-mediated suppression.

14. A method for making a genetically modified plant with improved grain digestibility, the method comprising:
a) transforming a cell with an expression cassette comprising an isolated nucleic acid selected from the group consisting of:
i) a polynucleotide of from about 21 nucleotides to about 40 nucleotides encoding an RNA that mediates RNA interference of an mRNA encoded by a polynucleotide comprising a nucleotide sequence of claim 1; and
ii) a polynucleotide encoding a transcript comprising a sense strand comprising the polynucleotide of (a), and an antisense strand comprising the complement of the polynucleotide of (a), wherein the transcript encoded by the polynucleotide is capable of forming a double stranded RNA;
b) regenerating a genetically modified plant from the cell; and
c) selecting for a genetically modified plant with improved digestibility.

15. An isolated polynucleotide of from about 21 nucleotides to about 40 nucleotides encoding an RNA that mediates RNA interference of an mRNA encoded by a polynucleotide comprising a nucleic acid of claim 1.

16. An isolated ds RNA capable of inhibiting expression of any one of the nucleic acid molecules of claim 1.

17. A method for making a genetically modified plant with seed having improved nutritional quality as compared the corresponding unmodified plant, the method comprising:
a) introducing into a plant cell an expression cassette with means for increasing the level of expression of a nucleotide sequence comprising at least one of the nucleotide sequences of claim 1;
b) regenerating a genetically modified plant from the cell; and
c) selecting for a genetically modified plant with seed having improved nutritional quality.

18. The method of claim 17 wherein the plant is selected from the group consisting of: soybean, alfalfa, or cassaya.

19. The method of claim 17 wherein the increased level of expression is directed to the seed or other storage organ or tissue of the plant.


SEQUENCE LISTING (23 sequences)

SEQ ID NO: 1 - DNA, 640 bp, Sorghum bicolor
  misc_feature (60)...(640): CDS
caccttcatctaaacaacagttagatctcaccatctgcaggatagtataatcaaataccatggcatcacaaacaaccaaaatgcttgccctggttgctgcgctccttgctctctctacgattgctactgccacagccaattgcctacaaaacatcccacatgtgatgggaatgacagtgatggatccatgcatgcagtcatgcatgatgcaacaaccattagccatggtaatgatgggaatgacagcgatggatccatgcatgcaatcatgcatgatgcaacaaccattagccatggtaataagttcaccctccctgatgatgagaatgaattccatggtctcgtgcgtgcaatcttgcatgacacaacgggcattctccataggaggctcatcattgtcaaaaatggtgatgcagccacaaccattctccatgatgcaacaacagtgctgcatgcaaccaatgatgatgcagggtctgatgtcaccaccgtgccactgtggtgctatgtgccagctgatgatgcacttgcaacagatgcaaatgacgctacaactgccttccatgtatagcactacagccatgccaactctagtggcctatggccaaaagccctttccttgttgtgcattctagataga

SEQ ID NO: 2 - protein, 192 aa, Sorghum bicolor
MASQTTKMLALVAALLALSTIATATANCLQNIPHVMGMTVMDPCMQSCMMQQPLAMVMMGMTAMDPCMQSCMMQQPLAMVISSPSLMMRMNSMVSCVQSCMTQRAFSIGGSSLSKMVMQPQPFSMMQQQCCMQPMMMQGLMSPPCHCGAMCQLMMHLQQMQMTLQLPSMYSTTAMPTLVAYGQKPFPCCAFI

SEQ ID NO: 3 - DNA, 903 bp, Saccharum officinale
  misc_feature (49)...(903): CDS
aaacaacagttagatctcactatctgaacgattgtataatcaaataccatggcatcacaaacaaccataatgcttgccctggttgtgctgcttgctctctctacgattgctactgccacaaccactttcctacaaaatggcccacatgtgatgggaatgatagtgatggatccatgcatgcagtcatgcatgatgcaacagccattagcatgcatgcagtcatgcatgatgcaacaaccattaggcatgataataggttcaccctccctgatgatgagaataaattccatggtctcgtgcatgcaatcttgcatgatgcagcaagcgttctccatagggggctcatcattgccaacaatgatgatgcggcaacaactattctccatggtgcaacaacagtgctgcatgcaaccaatgatgatgcagggtgtgatgtcaccactgtgccactgtggcgctatgtgccagatgatgcaattgcaacagatgcgaatgacgccacaactgccttccatgtgtagcactatagccatgccaactctggaggcctatggccaaaagccctttccttgttgtgcattctagatagattgatatccatgtgctgcattgtattccaataataaggtctcacataccatcgtgtgtgattcaccagaaataaaatgttgaatatgatgcatatattatggtaactacattcagtaatgtctcctaatgttggtgccttaaaacgttagctaagaaccatgtctcatagttagcttgagatgctattcttctttctggctatatgatcaggtacttcaaaagtgcaaagataaaatgttctagctagccagggttcgaaagttggaaacccctccttctcccttatgcccaaagttcaagtgatatatggcc

SEQ ID NO: 4 - protein, 276 aa, Saccharum officinale
MASQTTIMLALVVLLALSTIATATTTFLQNGPHVMGMIVMDPCMQSCMMQQPLACMQSCMMQQPLGMIIGSPSLMMRINSMVSCMQSCMMQQAFSIGGSSLPTMMMRQQLFSMVQQQCCMQPMMMQGVMSPLCHCGAMCQMMQLQQMRMTPQLPSMCSTIAMPTLEAYGQKPFPCCAFIDYPCAALYSNNKVSHTIVCDSPEIKCICIYYGNYIQCLLMLVPNVSEPCLIVSLRCYSSFWLYDQVLQKCKDKMFLARVRKLETPPSPLCPKFKYMA

SEQ ID NO: 5 - protein, 26 aa, Sorghum bicolor
  SIGNAL (0)...(0)
MASQTTKMLALVAALLALSTIATATA

SEQ ID NO: 6 - DNA, 3676 bp, Sorghum bicolor
  misc_feature (203)...(3383): CDS
taaactccgccagcgcagaggcctcatccgtctccatcccgcccgccacctcactcactcgctcaccgcctccgccggtgcccgcgcgtgcgccatccccactccagctcgctctctccattccgccgggaccacgtcgatcgaagtgccaggagttgtggatttcatgtctgtgatcgtggcaccttaattggactccgatatgggttctgctgctactgagagcaatgacaccttgctgggcaatggagttgttgggattcttgctgagacttgtaacatgtgggaaaggagggcaccattaactccttcccattgtgctcgccttctgctcggaggagggaagaacggagctcgagtaaaccggattactgtgcagccaagcacaaagaggatacatcatgacgctcagtatgaggatgtaggatgtgagatttcagaagacctgtcagaatgcggtcttattataggcatcaaacaaccaaagttgcagatgattcttccagatagagcgtacgctttcttttcacacactcacaaggcccaaaaagagaatatgccactgttagataagatccttgaagaaagggtgtccttgtttgattatgagctaattgttggagatgatgggaaaaggtcgctagcatttgggaagtttgctggtagagctggcctgatagatttcttacatggtctcggacagcgatatttgagccttggatactcgactccatttctctctctgggacaatctcatatgtatccttcgctcgctgcagccaaggctgcagtcattgtcgttgcagaagagatagcaacatttggacttccatccggaatttgtccgatagtgtttgtgttcactggagttggaaacgtctctcagggcgcgcaggagatattcaagttattgccccatacctttgttgatgctgagaaacttcctgaaatttttcagggcaggaatctgtctaaacaatctcagtcaaccaagagagtatttcaactatatggttgtgttgtgacctctagagacatggtttcccacaaggatcccaccagacaatttgacaaaggtgactattatgctcatccagaacactacgcccctgtttttcatgaaaggattgctccatatgcatctgtcatcgtaaactgtatgtattgggagaagaggcttccaccattactgagtatggatcagttacagcaactgatggagactggttgtcctttggttggcgtttgtgacataacttgtgatattggaggttccattgaatttgtcaacaagagtacatcaatagagaggcctttcttccggtatgatccttctaataattcataccatgatgatatggaaggtgccggagtgatctgcttggctgttgacattctccctacagagttctctaaagaggcctcccaacatttcggaaacatattatctaaacttgttcctagtttggcctcagtgaagcaactggcagaacttccttcctacttgagaagagcttgcattgcacatgctggcagattaactccgttgtatgaatatatccctagaatgagaaatactatgatagatttggcmcccacaaaaacaaatccattgcctgacaagaagtacagcaccctggtatctctcagtgggcacctatttgataagttccttataaatgaagctttggacatcattgagacaggtggaggttcatttcacttggttagatgtcaagttggacagagcatggatgatatgtcatactcagagcttgaagtaggagcagatgatactgccacattggataaaattattgattccttgacttctctagctaatgaacatggtggagatcacgatgccgggaaagaaactgaattagctctgaagataggaaaagtcaatgagtgtgaaactgatgccacagttgataaaggaggtccaaaggttttaattcttggagctggaagagtttgtcgaccagctgctgagtttctggcatcttacccaaacatatgtacctatggtgttgatgaccataacacagatcaaattcatgttattgtggcatctttgtatcaaaaagatgcagaagagacagttgatggtattgaaaatacaactgctacccagcttgatgtttctgatattggaagcctttcagatcttgtttctcaggttgaggttgtaattagcttgctgcctactagttttcatgctgccattgcaagagtatgcatagagctcaagaagcacatggtaacggcaagctatgttgatgaatccatgtcaaacttgagccaagctgctaaaggtgcaggtgtaactatactttgtgaaatgggcctagatcctggcatagatcacttgatgtcaatgaagatgattgatgaagctcatgctcgaaagggaaaaataaagacatttacatcttactgtggtggattgccatctccagctgcagcaaacaacccacttgcctataaattcagttggaacccagctggtgccctccgggcagggaaaaatcctgctgtctacaaatttcttggagagacgatccatgtagatggtcataacttgtttgaatcagcaaagaagctcagactaccagagcttccagcttttgctctggaacacttgccaaatcggaattccttgatatatggagacctttatggtatctccaaagaagcatctaccatatacagggctactcttcgttacgaaggttttagtgagataatggcaaccctgtcgaaaattgggttgtttgatgctgcaaatcatccactgctgcaagaaactaatcgcccaacatataagggtttccttgatgaactacttaataatatctccacaactaacacaggcttagatattgaagcctctggtggatatgatgatgaaatcattgccagactgtcgaagctcgggtgttgcagagataaggaaatagctgctaagacagtcaaaaccatcaagttcttgggactacatgaagagactcaaattcctaagggttgttcaagtgcatttgatgtgatttgccaacgaaaggaacagaggatggcctatggccacaatgagcaagacatggtactgctccaccacgaagttgaggtggaatacccagacgggcaacccaccgagaagcaccaagcgacgctactggagttcgggaaggttgaaaatggcagatccaccactgccatggccctgaccgttggtattccagcagcagtaggagccctgctattgctacagaataaggtccagacaaaaggagtgatcaggcctctgcaaccggagatctacattccagcattggagatcttggagtcgtcaggcatcaagctgattgagagtgtggagatttgatagttccgttcggatcaacttgctgtggctgtggctgcgctgcgctgcagccgtagcaactcccataagttcatcaaacgcaagctacagtgaaacggctgcaaggtagctacagtgacatggctgcgatgtatatgatagtatagctgggtacatatatcttttgtacatagtatattacctgtcgtaagagagcaaagaaacctctgtatatatgaccaaataatcccggcaaagaaacctatgtatatatatgatctatcaaataatgccgatctatattggctggct

SEQ ID NO: 7 - protein, 1060 aa, Sorghum bicolor
  VARIANT 464: Xaa = Any Amino Acid
MGSAATESNDTLLGNGVVGILAETCNMWERRAPLTPSHCARLLLGGGKNGARVNRITVQPSTKRIHHDAQYEDVGCEISEDLSECGLIIGIKQPKLQMILPDRAYAFFSHTHKAQKENMPLLDKILEERVSLFDYELIVGDDGKRSLAFGKFAGRAGLIDFLHGLGQRYLSLGYSTPFLSLGQSHMYPSLAAAKAAVIVVAEEIATFGLPSGICPIVFVFTGVGNVSQGAQEIFKLLPHTFVDAEKLPEIFQGRNLSKQSQSTKRVFQLYGCVVTSRDMVSHKDPTRQFDKGDYYAHPEHYAPVFHERIAPYASVIVNCMYWEKRLPPLLSMDQLQQLMETGCPLVGVCDITCDIGGSIEFVNKSTSIERPFFRYDPSNNSYHDDMEGAGVICLAVDILPTEFSKEASQHFGNILSKLVPSLASVKQLAELPSYLRRACIAHAGRLTPLYEYIPRMRNTMIDLXPTKTNPLPDKKYSTLVSLSGHLFDKFLINEALDIIETGGGSFHLVRCQVGQSMDDMSYSELEVGADDTATLDKIIDSLTSLANEHGGDHDAGKETELALKIGKVNECETDATVDKGGPKVLILGAGRVCRPAAEFLASYPNICTYGVDDHNTDQIHVIVASLYQKDAEETVDGIENTTATQLDVSDIGSLSDLVSQVEVVISLLPTSFHAAIARVCIELKKHMVTASYVDESMSNLSQAAKGAGVTILCEMGLDPGIDHLMSMKMIDEAHARKGKIKTFTSYCGGLPSPAAANNPLAYKFSWNPAGALRAGKNPAVYKFLGETIHVDGHNLFESAKKLRLPELPAFALEHLPNRNSLIYGDLYGISKEASTIYRATLRYEGFSEIMATLSKIGLFDAANHPLLQETNRPTYKGFLDELLNNISTTNTGLDIEASGGYDDEIIARLSKLGCCRDKEIAAKTVKTIKFLGLHEETQIPKGCSSAFDVICQRKEQRMAYGHNEQDMVLLHHEVEVEYPDGQPTEKHQATLLEFGKVENGRSTTAMALTVGIPAAVGALLLLQNKVQTKGVIRPLQPEIYIPALEILESSGIKLIESVEI

SEQ ID NO: 8 - DNA, 30 bp, Artificial Sequence
  a-kafirin B1 primer 1372
aagcttccatggctaccaagatacccgtcc

SEQ ID NO: 9 - DNA, 29 bp, Artificial Sequence
  a-kafirin B1 primer 1373
ggatccctatgtaagcctgcagagccggg

SEQ ID NO: 10 - DNA, 31 bp, Artificial Sequence
  a-kafirin A1 primer 1374
agatctgcactgctcacaaatttctatagct

SEQ ID NO: 11 - DNA, 26 bp, Artificial Sequence
  a-kafirin A1 primer 1375
tgtacacagctgcaaataggcagcgg

SEQ ID NO: 12 - DNA, 26 bp, Artificial Sequence
  a-kafirin B2 primer 1376
ggtacctaccgcttacttgcaacagc

SEQ ID NO: 13 - DNA, 36 bp, Artificial Sequence
  a-kafirin B2 primer 1377
gtcgactctaaaaaatggcaccaccaatgatgggtt

SEQ ID NO: 14 - DNA, 36 bp, Artificial Sequence
  d-kafirin 2 primer 1378
ctcgagctcgtgcgtgcaatcttgcatgacacaacg

SEQ ID NO: 15 - DNA, 26 bp, Artificial Sequence
  d-kafirin 2 primer 1379
agatctatgcacaacaaggaaagggc

SEQ ID NO: 16 - DNA, 22 bp, Artificial Sequence
  g-kafirin 1 primer 1380
ggatcctgcggcgttggcggtg

SEQ ID NO: 17 - DNA, 26 bp, Artificial Sequence
  g-kafirin 1 primer 1381
caattggaagcgcagggacttggctg

SEQ ID NO: 18 - DNA, 24 bp, Artificial Sequence
  g-kafirin 2 primer 1382
gaattcctcaggcaacagtgcaac

SEQ ID NO: 19 - DNA, 36 bp, Artificial Sequence
  g-kafirin 2 primer 1383
gtcgacccgggataatttaactgtgaaaaccacaag

SEQ ID NO: 20 - DNA, 31 bp, Artificial Sequence
  Sorghum LKR primer 1402
accggtgcaatgacaccttgctgggcaatgg

SEQ ID NO: 21 - DNA, 31 bp, Artificial Sequence
  Sorghum LKR primer 1403
gtcgacccgggataagaccgcattctgacag

SEQ ID NO: 22 - DNA, 33 bp, Artificial Sequence
  d-kafirin 2 pre-pro ppt primer forward
gtataatcacccgggatggcatcacaaacaacc

SEQ ID NO: 23 - DNA, 27 bp, Artificial Sequence
  d-kafirin 2 pre-pro ppt primer reverse
ggatatatcaagatctctagaatgcac